(19) United States
(12) Patent Application Publication
(10) Pub. No.: US 2025/0177972 A1
(43) Pub. Date: Jun. 5, 2025

(54) INTEGRATED ANALYSIS SYSTEM FOR THE RAPID MEASURING AND RECORDING OF DATA FROM A HOST

(71) Applicant: Hugh Califf, Morton Grove, IL (US)

(72) Inventor: Hugh Califf, Downers Grove, IL (US)

(21) Appl. No.: 18/530,167

(22) Filed: Dec. 5, 2023

(51) Int. Cl. B01L 3/00; A61B 10/02

(52) U.S. Cl. CPC B01L 3/502715; A61B 5/0002; A61B 5/02; A61B 5/145; A61B 10/0233; B01L 2300/023; B01L 2300/045; B01L 2300/0663; B01L 2300/12; G01N 33/50

(57) ABSTRACT

An integrated analysis system for the rapid measuring and recording of data from a host is provided. The system includes a flow chamber for quantitative measurements, sensors, memory cartridges, and at least one remote access device. The flow chamber is configured to have at least one microtube and at least one medical diagnostic sensor and wherein the medical diagnostic sensor is configured for measuring and monitoring of the biological materials of a host. The AI processing units integrated into the system allow for the exchange of data between the components of the of the flow chamber and a remote access device.

### RELATED APPLICATIONS

This application claims priority to U.S. Provisional Patent Application Ser. No. 63/431,107 filed Dec. 8, 2022, now pending.

### FIELD OF THE INVENTION

The present disclosure is directed towards the construction and design of an integrated analysis system for the rapid measuring and recording of data from a host. More particularly, the subject matter is the means by which artificial intelligence (hereinafter AI) supports the tracking, measuring, and monitoring of biological materials of a host using at least one medical diagnostic sensor in the integrated analysis system.

### BACKGROUND OF THE INVENTION

In the field of medical diagnostics, there is an increasing demand for accurate, rapid, and efficient systems capable of measuring and recording data from biological materials for tracking, measuring, and monitoring purposes. Conventional methods often involve time-consuming processes and may lack the ability to provide real-time and in-depth analysis of biological samples.

To address these challenges, various sensors and diagnostic tools have been developed to measure specific parameters of biological materials, such as density, flow rate, cellular features, and oxygen levels. Additionally, advancements in artificial intelligence have provided opportunities to enhance the analysis and interpretation of complex datasets derived from biological samples.

Furthermore, the integration of memory storage devices with analysis systems has become crucial for efficiently storing and accessing large volumes of data generated during the measurement and recording processes, enabling subsequent retrieval and analysis.

### SUMMARY

In order to accomplish the objectives of the present disclosure, there is provided An integrated analysis system for the rapid measuring and recording of data from a host comprising a flow chamber for quantitative measurements, sensors, memory cartridges attached to the flow chamber, and at least one remote access device.

In an embodiment, a flow chamber having at least one microtube and at least one medical diagnostic sensor embedded therein utilizes at least one medical diagnostic sensor for the in vivo and in vitro tracking, measuring, and monitoring of the biological materials of the host. AI processing units integrated into the system allow for the exchange of data between the memory cartridges, sensors, and AI processing units. At least one of the sensors is adapted for placement in a flow chamber and being interconnected and adapted for wireless communication with a remote access device that is configured for AI verification of the in vivo and in vitro tracking, measuring, and monitoring of the biological materials

Unless otherwise defined, all technical and scientific terms used herein have the same meaning as commonly understood by one of ordinary skill in the art. Although methods and materials similar or equivalent to those described herein can be used in the practice or testing of any described embodiment, suitable methods and materials are described below. In addition, the materials, methods, and examples are illustrative only and not intended to be limiting. In case of conflict with terms used in the art, the present specification, including definitions, will control.

The foregoing summary is illustrative only and is not intended to be in any way limiting. In addition to the illustrative aspects, embodiments, and features described above, further aspects, embodiments, and features will become apparent by reference to the drawings and the following detailed description and claims.

### BRIEF DESCRIPTION OF THE DRA WINGS

The present embodiments are illustrated by way of the figures of the accompanying drawings, which may not necessarily be to scale, in which like references indicate similar elements, and in which:

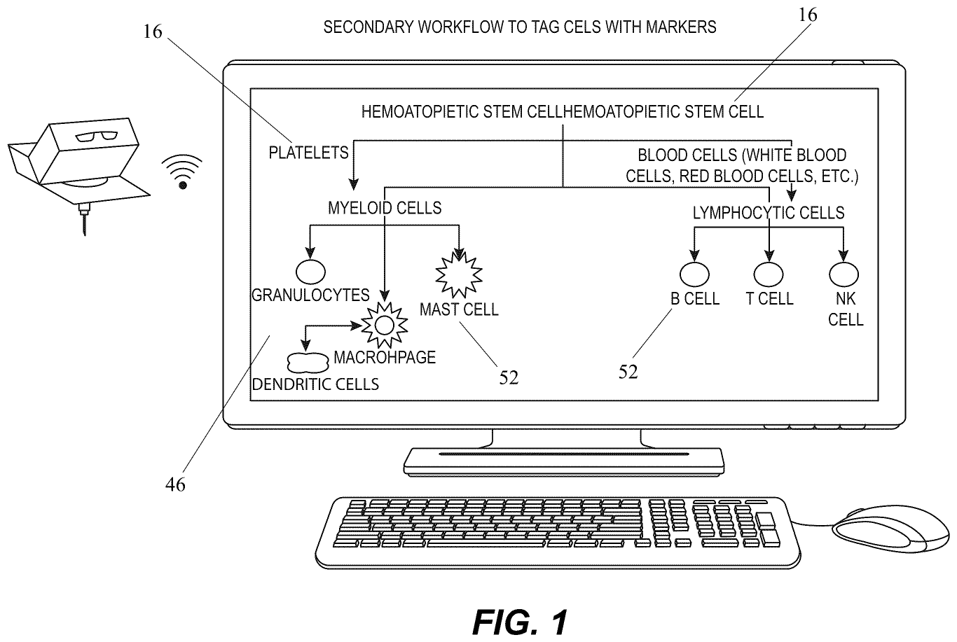

FIG. 1 is a hematologic analysis example of a complete blood count;

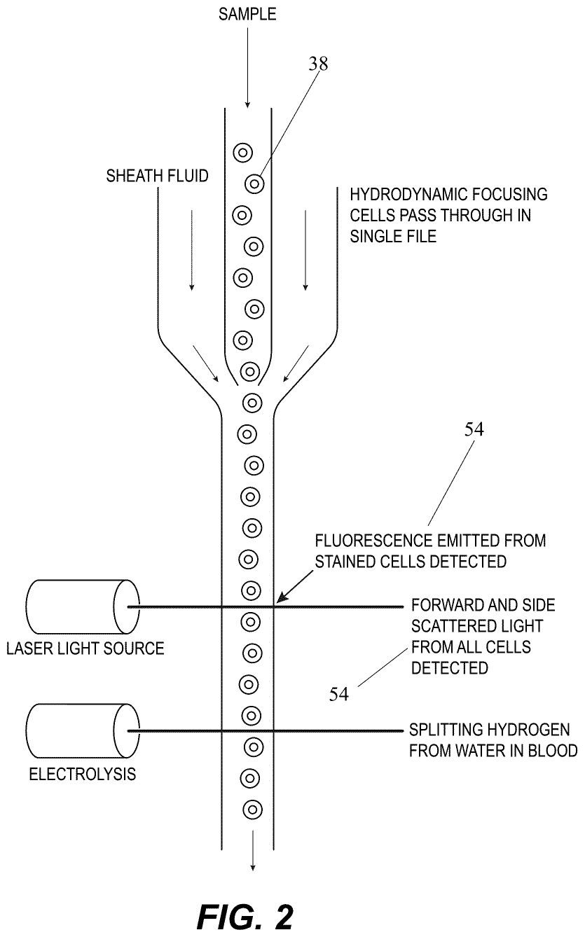

FIG. 2 is a secondary cellular analysis to detect the presence of a certain type of immune cell;

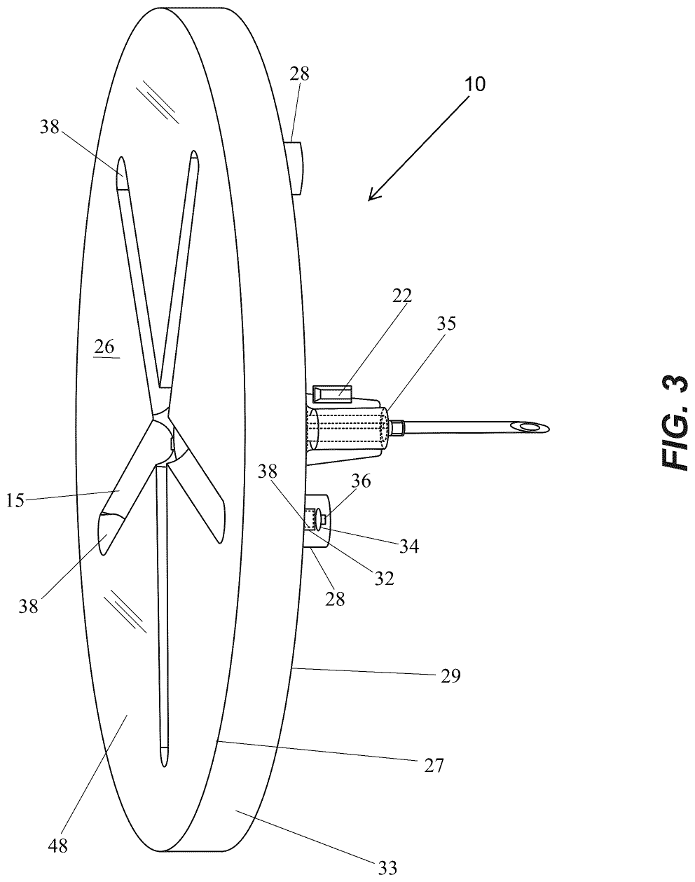

FIG. 3 shows the arrangement of the flow chamber and at least one microtube;

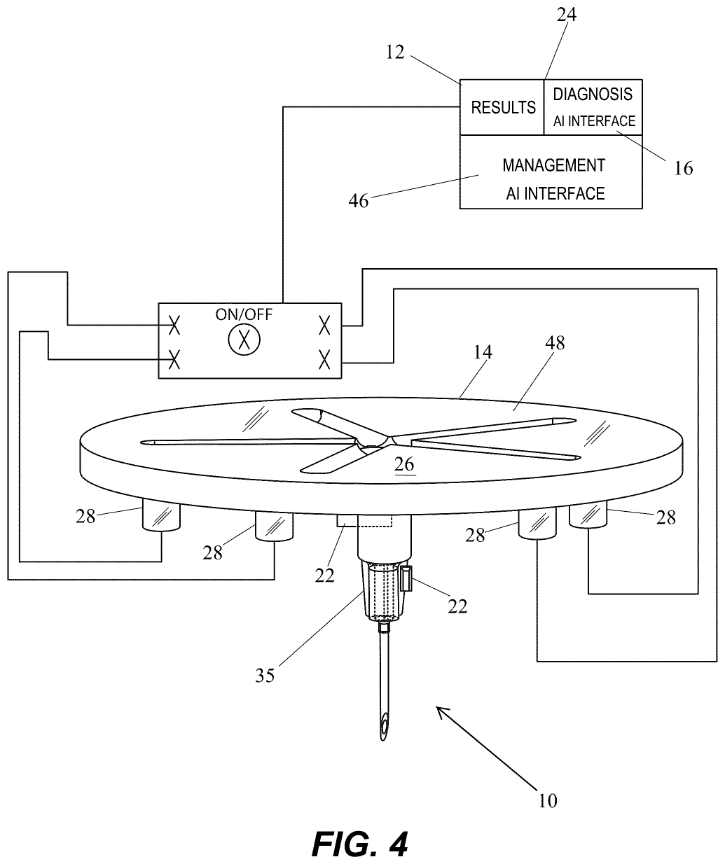

FIG. 4 shows the physical electronic connections between the flow chamber, sensors, memory cartridge, and an external monitoring device;

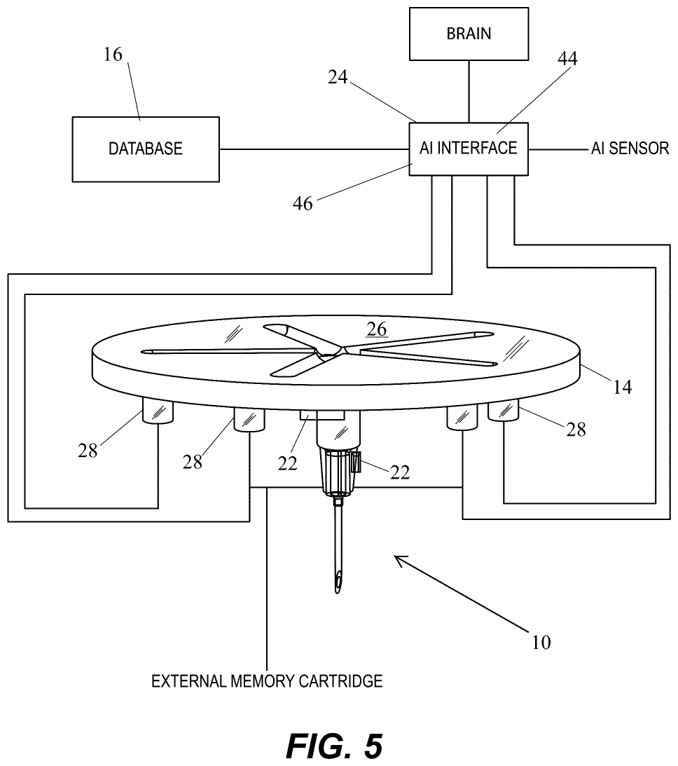

FIG. 5 shows the interplay of sensors used by AI, external monitoring devices, and physical electronic connections between the flow chamber, flow chamber sensors, memory cartridge, and an external monitoring device;

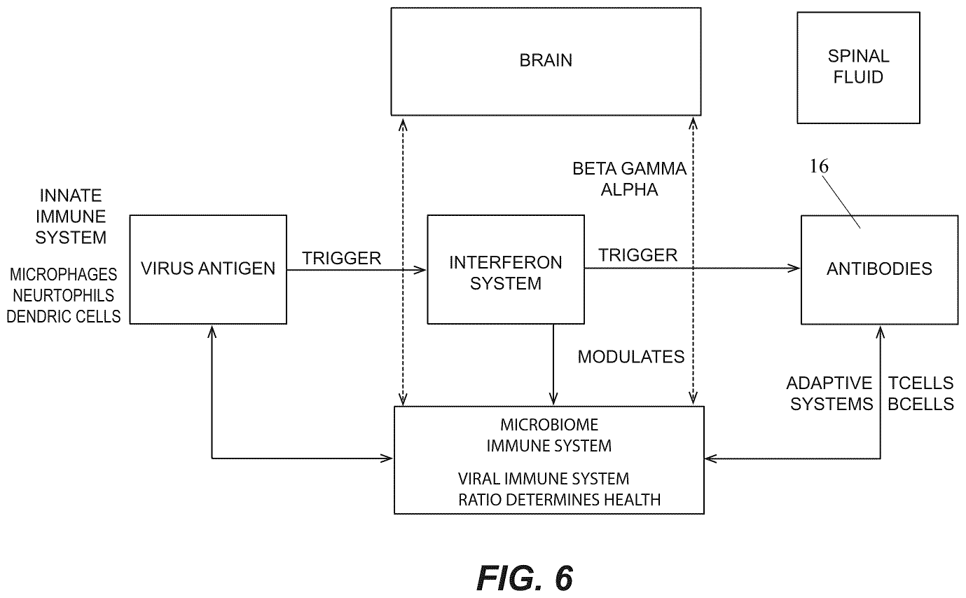

FIG. 6 shows an exemplary AI learning possibility of the positive or negative influences that inflammation can have on the microbiome and the impact of these influences on the brain;

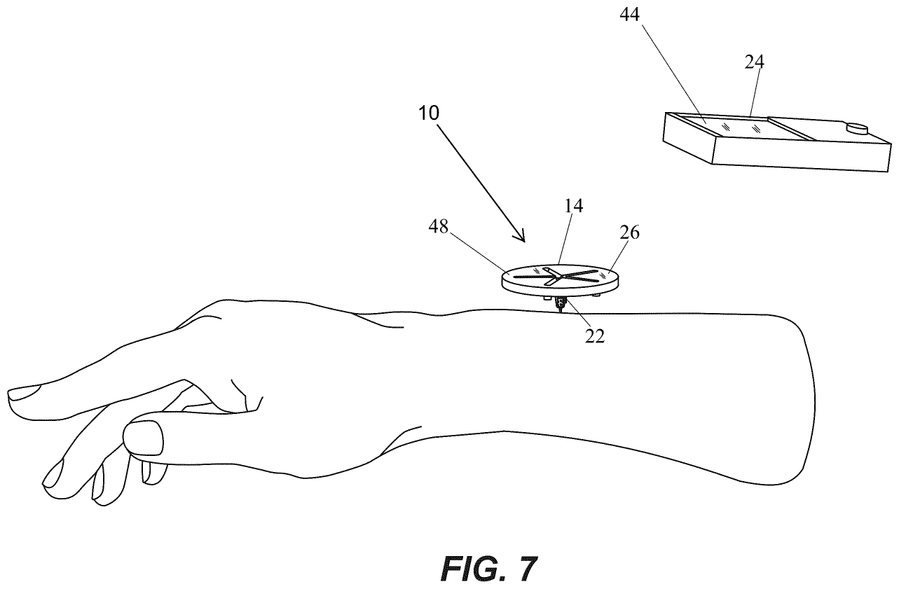

FIG. 7 shows an exemplary biological material collection method using the flow chamber and the proximity of an external monitoring device;

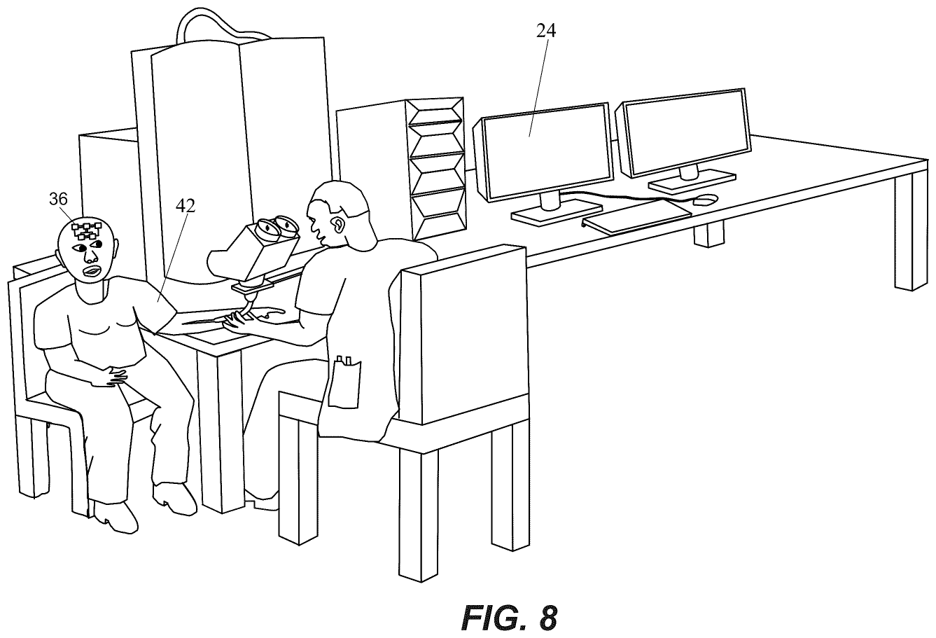

FIG. 8 shows an exemplary biological material collection method using the flow chamber and the proximity of an external monitoring device; and

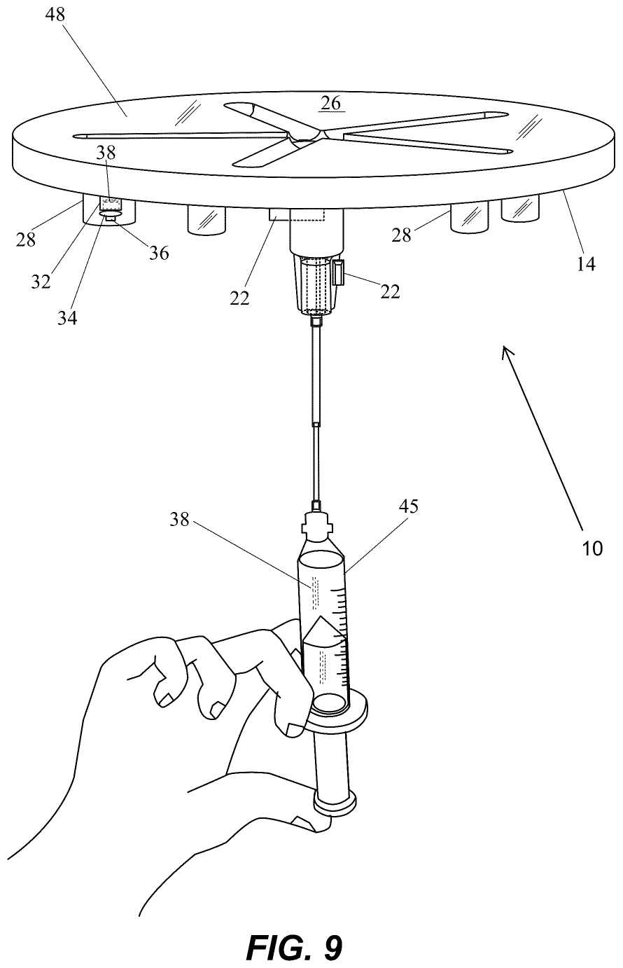

FIG. 9 shows an exemplary biological material collection method using a clinical specimen collection device to load the flow chamber.

### DETAILED DESCRIPTION

The following detailed description is the best-contemplated mode of carrying out the disclosure. Although the disclosure has been explained in relation to its preferred embodiment, it is to be understood that many other possible modifications and variations can be made without departing from the spirit and scope of the invention.

Therefore, the integrated analysis system 10 may be used in various types of settings including hospitals, clinics, physician offices or in remote areas such as military situations, remote hospitals with limited transportation or staff mobility, emergency situations near the site of an accident, or the same where there are limited resources to perform a quick diagnostic workup with immediate results. The device is designed to rapidly deliver information using a minimal amount of blood or other biological materials 38, thereby enabling timely results. This capability is particularly valuable in emergency room settings or similar environments where prompt results are essential for delivering appropriate medical care.

The flow chamber 14 of the integrated analysis system 10 can include various medical diagnostic sensors 36 including electrochemical sensors, optical sensors, impedance-based sensors, or the like to measure various parameters 52 such as analytes, blood gases, oxygen saturation, hematocrit levels, and other components once the sensor 36 is exposed to biological materials 38 such as blood, urine, spinal fluid, saliva, respiratory fluids, or the same. Syringes, butterfly needles or the like can function as the clinical specimen collection equipment as provided herein. If desired by the healthcare facility, skin puncture of a host 42 by the needle that extends through the flow chamber 14 can be administered to facilitate the collection of the biological material 38.

The remote access device 24 of the system 10 as provided herein can include a cell phone, computer, or similar device for capturing and communicating the signals from the sensors 36.

### Quantitative Measurements

In a preferred use of the integrated analysis system 10 and as shown in FIGS. 1-9, the integrated analysis system 10 for the rapid measuring and recording of data 12 from a host 42 comprises a flow chamber 14 for quantitative measurements 16, sensors 36, memory cartridges 22 attached to the flow chamber 14, a remote access device 24. The flow chamber 14 having a top surface 26 and at least one flow channel 28 connected to at least one microtube 32 with a compartment 34 for receiving at least one medical diagnostic sensor 36 therein and wherein the at least one medical diagnostic sensor 36 is configured for the in vivo and in vitro tracking, measuring, and monitoring of the biological materials 38 of the host 42. The integrated analysis system 10 uses standard electrochemical principles for measuring and recording the data 12 once the biological material 38 is introduced into the flow channel 28.

AI processing units 44 are integrated into the system 10 through interfaces 46 that allow for the exchange of data 12 between the memory cartridges 22, sensors 36, remote access device 24, and AI processing units 44. At least one of the sensors 36 is adapted for connection to a host 42 and at least one of the sensors 36 is adapted for placement in the flow chamber 14 and interconnectivity and wireless communication with the remote access device 24, and wherein the remote access device 24 is configured for AI verification of the in vivo and in vitro tracking, measuring, and monitoring of the biological materials 38. The system 10 can be configured for additional measuring and recording of data 12 from a host 42 with the exchange of the data 12 and with technology advances. Integrating advanced technology can lead to more accurate testing, high-speed analysis of data 12 to help identify trends, and comprehensive results.

The integrated analysis system 10 further provides for the flow chamber 14 to removably connect to a clinical specimen collection equipment further providing for the collection of biological materials 38 in the microtube 32. In addition to syringes, various types of clinical specimen collection equipment typically used in healthcare setting may be used. This can include blood collection tubes, vacutainers, swabs, specimen containers, needles, or the like for the proper collection, preservation, and transport of samples for diagnostic testing. The flow chamber 14 further including a glass slide 48 that is horizontally extending across the top 26 and removably fixed thereon to cover the biological materials 38 contained within the microtube 32. When selecting a cover material for the flow chamber 14, the user of the device an consider use additional materials such as quartz, plastic, silicone coverslips, or similar materials which offers optical properties, chemical resistant, and durable.

The at least one remote access device 24 enables the remote monitoring and control of the analysis system 10. The sensor 36 integrated within the microtube 32 is configured to measure one or more parameters 52 of the biological material 38, including but not limited to, concentration, flow rate, or cellular characteristics. The sensor 36 is in direct contact with the biological material 38 in order to accurately measure the desired parameter with the measured signals 54. Measured signals can include scattered light measurements, fluorescence, or changes in properties being processed and interpreted by the remote access device 24 to provide a quantitative measurement 16 of the parameter being assessed.

### Measuring, Recording, and Monitoring Data

The system 10 as provided herein is designed to provide preliminary or detailed information that is reliable and can immediately guide decisions in emergency situations. For example, in the case of a trauma patient where blood count is immediately required, the device can be used to determine the need for a blood transfusion or at least extra volume administration of additional blood or crystalloid fluids. If more than one microtube 32 is being used and multiple parameters 52 are being measured, AI in cooperation with the history of the patient, can provide early examination findings. AI could help locate a differential diagnosis, provide the type of therapeutic recommendations and management that are needed, and provide all of the information that is available including the test results from the sensors 36. These monitoring steps can be done on the scene and a first-responder can initiate the process if additional medical support is not available. This system 10 would allow the first-responder to embark on diagnostics and initial medical management with the sensor information and the AI that is available. For example, the recommendations can be to supply blood and fluids, improve breathing support, of provide ventilation. Cardiac, respiratory, or some trauma related etiology can be considered.

In the case of an unconscious patient, the system 10 can recommend a blood gas analysis to determine how much respiratory support or fluid the patient needs. The system 10 can guide the immediate resuscitation or fluid decisions by providing the blood gas analysis including the partial pressure of carbon dioxide, partial pressure of oxygen, and base excess to provide an indication of lungs status and the metabolic status of the body. Whole blood diagnostics can include blood type determination and evaluation, hematologic concerns including the complete blood count, biochemical analysis including electrolytes and liver function measurements, biomarkers including C-reactive protein, pro calcitonin, and interferon.

### Biological Materials

In clinical settings, blood, spinal fluid, and other biological materials 38 for sampling are usually collected from hosts 42 such as human patients for diagnostic purposes. However, the system 10 as provided herein can apply to diagnostic testing in clinical settings, disease monitoring or research in veterinary settings, biomedical research on non-human primates, or any organism that harbors pathogens or parasites.

The type and amount of biological material 38 including whole blood or spinal fluid that is placed in a microtube 32 can include a fraction of a CC (0.1-0.3 CC) but this amount can be reduced or increased depending on the medical response scenario and setting. For example, bacterial or viral pathogens can be detected on a small sample using integrated PCR technology. A similar sensor can be used to detect a bacterial match in a blood sample. A sensor of the type used in PCR technology is prepared with a certain film that can detect certain viruses or bacteria. Smaller samples of biological materials 38 are also used for non-invasive prenatal DNA testing. A mother's blood can be used to determine the risk that a fetus will be born with certain abnormalities. This testing analyzes small fragments of cell-free DNA that are circulating in a pregnant mother's blood.

### Hematological Analysis (Secondary Analysis)

FIG. 1 shows that further analysis might be required after the preliminary or in-the-field analysis of the system 10 as provided herein. For example, further hematologic analysis at a core lab for a complete blood count can include white blood cells, hematocrit, hemoglobin, and platelet count. After a complete labeling of the biological materials 38 for further diagnosis and immunological analysis, a differentiation of the white blood cells might be required for a sample of the biological material 38 and a flow cytometry process can further automate the results. AI, learning from input 12 to provide new output data and results, learns over time if the results are accurate. This would depend on the algorithms and AI can then use the date to provide additional conclusions and interpretations.

FIG. 2 provides an additional example of a secondary cellular analysis to detect the presence of a certain type of immune cell to be able to discern the various distributions of immune cells in a sample. FIG. 3 shows the connection between the brain, microbiome, and GI system that have been studied over the past 20 years. FIG. 6 shows that, depending on a specific algorithm, AI programming can learn the positive or negative influences that inflammation can have on the microbiome and the impact of these influences on the brain. If there is inflammation then there is an inflammatory response in the GI system that can cause inflammation in the brain. Increased levels of interferon are released form the cells into the blood and those markers can have a natural flow influence on the brain cells. Spinal fluid can be used to detect the possibility of infection or inflammatory processes affecting the brain and sometimes found along the nerve fibers. The alternative to the system 10 provided herein would be a brain biopsy. Mediators, pro-inflammatory, or anti-inflammatory proteins released by immune cells in response to an infection can have an impact on the local body environment such as the bowels and also on remote organs that are not in the local environment of the body. The brain-intestine axis comes into play and nerve signaling has an impact on inflammation in the brain. For example, these abnormalities in the microbiome of a baby can be a contributing factor to some of the developments or neuro-developmental problems might be visible. A brain bleed is not always the contributing factor. It may be more indistinct through the abnormal balance of the biome causing inflammatory responses that create scaring in the brain tissues or influences even the development of the synapsis in the brain.

### Flow Chamber

Overall, the design of the flow chamber 14 and the microtube 32 used to hold a sensor 36 and biological materials 38 is aimed at providing a convenient and reliable repository for the measurement of specific parameters 52 in such materials, while ensuring safety, reliability, accuracy, and ease of use in a clinical or point-of-care setting.

The flow chamber 14 and associated microtubes 32 can be constructed in various sizes and can be provided as standard construction including the provision of durable materials for all elements as provided herein. It can be produced in a circular shape, a standard box-like shape or provided as desired by a particular healthcare facility. In a healthcare setting, the shape of a medical device plays a crucial role in facilitating easy and efficient handling of biological materials such as blood. The device's ergonomic design ensures that it can be comfortably held, operated, and easily integrated into the workflow of a healthcare professional.

The design of the flow chamber 14 and microtubes 32 can vary depending on the specific application and the type of sensors 36 being used and can me made of biocompatible materials to ensure that the materials do not alter the composition of the biological materials 38. Common materials might include medical-grade plastics or polymers. The flow chamber 14 and microtubes 32 can be designed to be disposable and single-use to prevent cross-contamination and ensure the accuracy of subsequent measurements.

The microtube 32 may include features such as capillary action channels or microfluidic pathways to enable the efficient movement of the biological material 38 to the sensor 36. The microtube 32 includes a compartment 34 where the sensor 36 is inserted and the sensor 36 can be positioned to have direct contact with the biological materials 38 and thereby allowing for the measurement of the desired parameter. The design of the microtube 32 ensures that the sensor 36 remains securely in place and properly aligned to enable accurate measurements. In some cases, the microtube 32 may include electrical contacts, optical windows, or other interfaces 46 that allow the sensor 36 to communicate with external measurement or analysis equipment. The microtube 32 may also include features to ensure proper alignment and connection with the measurement device or instrument.

The protective cover is a safety feature that prevents accidental exposure to the biological material 38. Additional safety features such as tamper-evident seals for the chamber 14 and the sensor compartment 34 may also be incorporated to minimize the risk of exposure to biohazards during the handling and disposal of the microtube 32. The chamber 14 and the microtube 32 may also include markings, labels, or identifiers to convey important information such as the type of sensor, lot number, expiration date, and any specific handling instructions.

### Artificial Intelligence (AI)

Depending on the specific requirements and constraints of a facility, AI could be integrated into the system 10 through interfaces 46 that allow for the exchange of data 12 between the memory cartridges 22, sensors 36, and AI processing units 44. This may involve wireless or wired connections and integration of AI into the evaluation system 10. It has the potential to enhance the accuracy, efficiency, of not just the analysis of the biological material 10 but also the effectiveness of medical diagnostics and patient care. For example, AI can be important for data analysis and identifying patterns, anomalies, and trends in the biological sample data 12. This analysis could help in diagnosing medical conditions, predicting patient outcomes, or providing insights for medical research. In addition, AI could boost and reinforce the medical conclusions of healthcare professionals by processing the data 12 and providing insights or recommendations based on the blood sample evaluation. This could further provide support for clinicians when constructing opinions with regards to a specific treatment method or plan. The user or healthcare professional can ensure that the use of AI applications adhere certain to regulatory standards, privacy considerations, and ethical guidelines.

### Memory Cartridges

Additional specific requirements for the mechanical, wireless, and electrical connections of the system 10 will depend on the design of the memory cartridge 22, the sensors 36, and the biological material evaluation. In a medical context, the user or healthcare facility must adhere to all relevant industry and regulatory standards for medical devices and to ensure the safety and effectiveness of the connections in a medical setting.

Certain components necessary to the operation of the memory device or other parts of the chamber 14 are not shown or described in detail because they are components well known to those in the relevant arts. These components include well-known components, attachments, parts, and operations. For example, a memory cartridge in a general sense is a storage device designed to hold digital data and the function of the memory cartridge 22 will vary depending on the specific application in a healthcare setting. A few common functions of the memory cartridge 22 as provided herein can include storage and retrieval of digital data such as files, documents, images, and software.

Physically connecting memory cartridges 22 to a chamber 14 and to a series of sensors 36 for evaluating samples of biological materials 10 involves a plurality of considerations regarding mechanical, wireless, and electrical connections. The design as provided herein considers the compatible physical dimensions and connections between a specific memory cartridge 22 and a chamber 14 to ensure a secure mechanical connection. Given the medical nature of the chamber 14, the mechanical connection should be durable and able to withstand repeated use and potential sterilization processes. The mechanical connection should be designed for easy insertion and removal to facilitate the workflow of biological material evaluation.

If wireless communication is used, the memory cartridge 22 and sensors 36 should support a reliable wireless protocol for transmitting data 12 to the remote access device 24. This could be Bluetooth, Wi-Fi, or another suitable wireless standard. If sensitive patient data 12 is being transmitted wirelessly, the connection should incorporate appropriate security measures to protect the data 12 from unauthorized access or interception.

The chamber 14, memory cartridge 22, and sensors 36 can have a consistent power supply, whether through batteries, charging, or another power source and the electrical connection can support the transfer of data 12 between the memory cartridge 22, the sensors 36, and ensuring that information from the biological material evaluation is accurately stored and accessible.

While the invention has been described in detail and with reference to specific embodiments thereof, it will be apparent to one skilled in the art that various changes and modifications can be made therein without departing from the spirit and scope thereof.

What is claimed is:

1. An integrated analysis system for the rapid measuring and recording of data from a host, the system comprising:
a fully enclosed flow chamber having an interior space for quantitative measurements and housing medical diagnostic sensors;
memory cartridges attached to the flow chamber, and at least one remote access device in wired and wireless communication with the flow chamber;
the flow chamber having a top surface with a circular periphery, a bottom surface, and a sidewall extending from the circular periphery and being integrally connected to the bottom surface to define the interior space within the flow camber;
the flow chamber including a passageway and a plurality of flow channels being fixedly attached to the bottom surface thereof;
the passageway being a substantially circular hollow tube provided on a substantially vertical axis for insertion of an aqueous biological material therein and into the plurality of flow channels, the flow channels having outer ends and being provided in an interconnected radial arrangement with respect to the passageway and beneath the top surface of the flow chamber and wherein the flow channels radially extend away from the passageway and being connected to a plurality of microtubes at the outer ends thereof;
wherein the plurality of microtubes having interior compartments for receiving the medical diagnostic sensors and the aqueous biological once the aqueous biological material is inserted into the passageway and flows through the flow channels thereof;
wherein the medical diagnostic sensors track the in vivo and in vitro measuring and monitoring of the aqueous biological material of the host using standard electrochemical principles for measuring and recording the data once the aqueous biological material is introduced into the flow channels;
wherein artificial intelligence processing units incorporating machine learning algorithms to analyze the data in real time are integrated into the memory cartridges and medical diagnostic sensors of the system through interfaces allow for the exchange of the data between the memory cartridges, medical diagnostic sensors, and artificial intelligence processing units and wherein the artificial intelligence processing units utilize predictive analytics to enhance the accuracy of measurements from the aqueous biological material;
wherein at least one of the medical diagnostic sensors being adapted for connection to the host; wherein the system measures and records additional data from the host; and
at least one of the medical diagnostic sensors being placed in the flow chamber and being interconnected for wireless and wired communication with the remote access device, and wherein the remote access device executes artificial intelligence verification of the in vivo and in vitro tracking, measuring, and monitoring of the aqueous biological material.

2. The integrated analysis system of claim 1, wherein the flow chamber is removably connected to a clinical specimen collection equipment provided for the collection of the aqueous biological materials in the plurality of microtubes.

3. The integrated analysis system of claim 1, wherein the flow chamber includes a glass slide horizontally extending across the top surface thereof and fixed thereon to enclose the radial arrangement of the plurality of flow channels, the passageway, the plurality of microtubes, medical diagnostic sensors and the aqueous biological material contained within the plurality of microtubes.

4. The integrated analysis system of claim 1, wherein the at least one remote access device enables remote monitoring and control of the integrated analysis system.

5. The integrated analysis system of claim 1, wherein the medical diagnostic sensors integrated within the microtubes are measure one or more parameters of the aqueous biological material, including concentration, flow rate, and cellular characteristics, and is in direct contact with the aqueous biological material in order to accurately measure the one or more parameter, with measured signals being processed and interpreted by the remote access device to provide a quantitative measurement of the one ore parameters being assessed.

6. (canceled)

7. The flow chamber of claim 1 wherein the plurality of microtubes of the flow chamber receive the aqueous biological material via the passageway.

8. (canceled)

9. (canceled)
